(19) United States
(12) Patent Application Publication
(10) Pub. No.: US 2010/0082066 A1
(43) Pub. Date: Apr. 1, 2010

(54) POSTERIOR FIXATION DEVICE FOR PERCUTANEOUS STABILIZATION OF THORACIC AND LUMBAR BURST FRACTURES

(76) Inventor: Ashok Biyani, Sylvania, OH (US)

Correspondence Address: MACMILLAN SOBANSKI & TODD, LLC, ONE MARITIME PLAZA FIFTH FLOOR, 720 WATER STREET, TOLEDO, OH, 43604-1619, US

(21) Appl. No.: 12/569,133

(22) Filed: Sep. 29, 2009

Related U.S. Application Data
(60) Provisional application No. 61/101,532, filed on Sep. 30, 2008.

(51) Int. Cl. A61B 17/70

(52) U.S. Cl. 606/250; 606/264

(57) ABSTRACT

A spinal fixation device includes a fixation rod including a body having an aperture formed therethrough. First and second vertebral fasteners are secured to the body and are adapted to be secured to respective vertebrae. A third vertebral fastener extends through the aperture in the body of the fixation rod and adapted to be secured to a vertebra.

### STATEMENTS REGARDING FEDERALLY SPONSORED RESEARCH OR DEVELOPMENT AND CROSS REFERENCE TO RELATED APPLICATIONS

This invention was not made with any government support. This application claims the benefit of U.S. Provisional Application No. 61/101,532 filed Sep. 30, 2008, the disclosure of which is incorporated herein by reference.

### BACKGROUND OF THE INVENTION

This invention relates to the field of orthopedic surgery and more particularly to the area of spinal surgery. Specifically, this invention relates to an improved fixation rod for use in stabilizing thoracic and lumbar spinal burst fractures and to a method for using same.

Fractures of the vertebrae of the human spine can range from mildly painful conditions to serious life-threatening situations. One basis for classifying such spinal fractures is whether they are stable or unstable. Generally speaking, a stable fracture is a fracture of a vertebra that does not cause any spinal deformity or neurological problems. Stable fractures of the thoracic and lumbar spine are frequently treated in a conservative manner without surgery. An unstable fracture, on the other hand, make it difficult for the spine to carry and distribute weight. Consequently, unstable fractures usually result in a spinal deformity (such as kyphosis) which may progress and cause further damage. Unstable spinal fractures of the thoracic and lumbar spine are frequently treated with surgical procedures. Typically, after appropriate decompression of the neural elements (if necessary), the spinal column is stabilized with anterior, posterior, or a combined antero-posterior fixation.

More recently, percutaneous stabilization has been recommended for such fractures. Posterior fixation relies partly on the ligamentotaxis principle for restoration of alignment, but correction of kyphotic deformity is typically inadequate with such fixation devices. Thus, it would be desirable to provide an improved fixation rod for use in stabilizing thoracic and lumbar spinal burst fractures and to provide a method for using same.

### SUMMARY OF THE INVENTION

This invention describes a modified percutaneous fixation rod for posterior fixation. The fixation rod includes a locking hole near its mid-portion that allows for the fixation of the pedicle in the fractured segment. The technique is suitable for minimally invasive spine surgery, but may be easily modified to fit open surgical procedures if desired. Initially, all pedicles are cannulated and guide wires are inserted, followed by placement of end pedicle screws utilizing well established minimally invasive surgical techniques. A rod of appropriate length is placed, but set screws are not inserted until after the center vertebra with an intact pedicle has been fixed. If the pedicle of the center vertebra is also fractured, this vertebral segment may be skipped and the assembly completed by connecting the longitudinal member to the vertebral anchors above and below the fracture site. Typically, at least one pedicle remains intact, which allows for fixation of the fractured vertebra for improved stability and alignment. Such a construct may also permit utilization of short segment fixation, such as one segment above and below instead of two vertebrae above and two below. When the pedicle is intact in the fractured segment, a guide wire is placed in a routine manner prior to insertion of any hardware. After end screw placement, the rod is inserted and held in place provisionally. The locking pedicle screw is placed through the hole in the rod. The mid portion of the rod, which contains the locking hole for fixation of the fractured vertebra, is thicker than the rest of the rod and acts as a fulcrum when a corrective force is applied as the end vertebrae are fastened to the longitudinal member. Locking screws also improve fixation and pullout strength of the construct. As the set screws are inserted in the end vertebral anchors, the kyphotic deformity of the fractured vertebra is corrected by the principles of ligamentotaxis and its height is restored. The invention also allows flexibility of skipping the fractured pedicle or insertion of locking and non-locking screw in the intact pedicle of a fractured vertebra.

This invention also describes modifications to existing techniques of posterior pedicle screw fixation for treatment of thoracolumbar burst fractures. The longitudinal member typically has a 5.5 mm or similar diameter rod configuration, identical to currently utilized constructs. This configuration permits surgeon familiarity with top loading vertebral fasteners, also know as pedicle screws.

The central portion of the longitudinal member, which overlays the fractured vertebra, incorporates a hole for insertion of a screw though the rod and into the pedicle of the fractured vertebra. The central hole through the rod for fixation to the fractured pedicle may have several shapes. It may be incomplete such that it allows realignment of the previously inserted guide wire. The guide wire is brought into the central hole through a laterally or medially based opening in the hole. An external tool, such a tube or cannula, may be placed over the guide wire to bring it over the rod such that a screw can be inserted through the hole within the rod. Following this, the end construct may be completed.

The rod may be inserted such that the opening in the central hole is lateral or medial, as dictated by patient anatomy and position of the guide wire. In another variation of the open ended central hole, a collar or a washer may be placed or slid over the lateral or medial opening to improve rigidity of the construct. In yet another variation, the rod may incorporate a central hole that is complete without medial or lateral openings for guide wire displacement into the hole. In this modification, the guide wire is placed in the fractured segment with intact pedicle, prior to rod insertion. The end vertebral anchors are placed next. The longitudinal member has a slit-like opening at its both ends that could extend (up to one centimeter or more) on either side of the central hole to accommodate the displacement of guide wire within the rod as it inserted. Such an opening allows the rod to be placed over the guide wire initially almost parallel to it before entering the skin and subcutaneous tissue. The rod is inserted as far distally as possible and then pulled back into the proximal most insertion tube.

After the locking hole has been centralized over the guide wire, the screw is inserted into the pedicle of the fractured vertebra. Typically, a screw of 25-45 mm in length can be inserted in the fractured vertebra. Once the rod has been provisionally secured to the locking hole, the end vertebrae are fastened to the longitudinal members. This maneuver improves spinal alignment and will also correct angular deformity as a result of ligamentotaxis. Final tightening is performed after all set screws have been inserted.

If desired, guide wires may be placed in the end vertebra only initially followed by placement of pedicle screws. A longitudinal member of the disclosed invention is then placed percutaneously or by utilizing an open technique. The pedicle of the fractured vertebra is then targeted through the central hole in the rod. A guide wire is then placed followed by insertion of the locking pedicle screw in the fractured vertebra though the central opening in the longitudinal member. Set screws are inserted in the end vertebral anchors last and assembly completed.

In yet another variation, the central hole through the rod may permit insertion of non-locking screws utilizing either sequence described above. Finally, if the pedicle is fractured, placing a pedicle screw at this level may not be advisable. In this setting, a “filler” locking screw may be inserted that fills the hole within the rod but does not anchor the vertebra below. This arrangement will improve the strength of the rod. The rod of such description may be designed to encompass more than one vertebra above or below the fracture level to suit the requirements of any given patient.

The vertebral fasteners for fixation to the intact vertebra above and below the fracture may be of any prior art and are typically top loading. The rod is designed to be able to fit any pedicle screw with 5.5 mm or 6.35 mm or similar diameter pedicle screws. The vertebral fastener for the fractures segment is inserted through a central complete or incomplete hole in the rod. There may be a collar or a washer under the screw if desired to bridge the lateral or medial opening. The vertebral fasteners may also incorporate a locking feature if desired such that the screw locks on to the rod improving its stability and spinal alignment. Finally, in case where pedicle screw fixation of the fractures segment is not desirable, a small screw may be inserted to fill the hole in the rod.

Various objects and advantages will become apparent to those skilled in the art from the following detailed description of the preferred embodiments, when read in light of the accompanying drawings.

### BRIEF DESCRIPTION OF THE DRAWINGS

FIG. 1 is a schematic side elevational view of three vertebrae of a portion of a human spine, wherein the upper and lower vertebrae are undamaged and the intermediate vertebra has experienced a burst fracture.

FIG. 2 is a schematic side elevational view similar to FIG. 1 showing a guide wire inserted into each of the upper, intermediate, and lower vertebrae.

FIG. 3 is a schematic side elevational view similar to FIG. 2 showing first and second pedicle screws inserted into each of the upper and lower vertebrae.

FIG. 4 is a perspective view of a first embodiment of a fixation rod in accordance with this invention.

FIG. 5 is a top plan view of the first embodiment of the fixation rod illustrated in FIG. 4.

FIG. 6 is a sectional elevational view of the first embodiment of the fixation rod taken along line 6-6 of FIG. 5.

FIG. 7 is a bottom plan view of the first embodiment of the fixation rod illustrated in FIGS. 4, 5, and 6.

FIG. 8 is a schematic side elevational view similar to FIG. 3 showing the first embodiment of the fixation rod in the process of being installed on the first and second pedicle screws.

FIG. 9 is a schematic side elevational view similar to FIG. 8 showing the first embodiment of the fixation rod preliminarily installed on the first and second pedicle screws.

FIG. 10 is a schematic side elevational view similar to FIG. 9 showing a locking screw inserted into the intermediate vertebra and secured to the first embodiment of the fixation rod.

FIG. 11 is a schematic side elevational view similar to FIG. 10 showing the first embodiment of the fixation rod finally installed on the first and second pedicle screws so as to correct the orientation of the vertebrae.

FIG. 12 is a schematic side elevational view similar to FIG. 11 of a first alternative embodiment of a locking screw in accordance with this invention.

FIG. 13 is a schematic side elevational view similar to FIG. 11 of a second alternative embodiment of a locking screw in accordance with this invention.

FIG. 14 is a schematic side elevational view similar to FIG. 11 of a filler screw that can be used in lieu of the locking screw in accordance with this invention.

FIG. 15 is a perspective view of a second embodiment of a fixation rod in accordance with this invention.

FIG. 16 is a top plan view of the second embodiment of the fixation rod illustrated in FIG. 15.

FIG. 17 is a sectional elevational view of the second embodiment of the fixation rod taken along line 17-17 of FIG. 16.

FIG. 18 is a bottom plan view of the second embodiment of the fixation rod illustrated in FIGS. 15, 16, and 17.

### DETAILED DESCRIPTION OF THE PREFERRED EMBODIMENTS

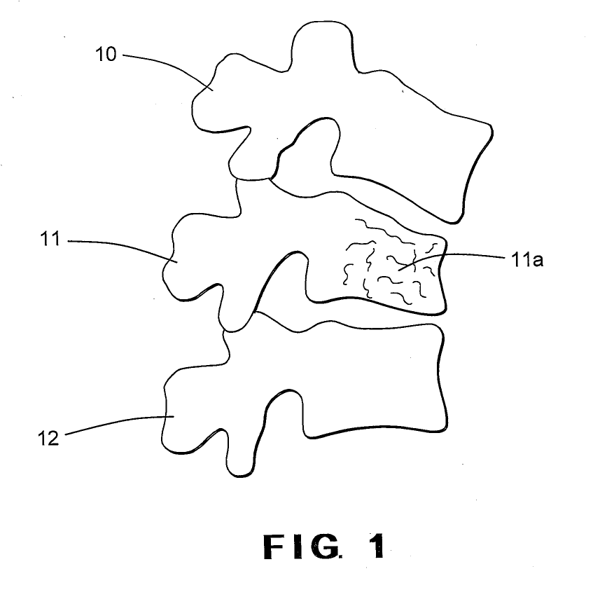

Referring now to the drawings, there is illustrated in FIG. 1 three vertebrae 10, 11, and 12 of a portion of a human spine. The illustrated vertebrae 10, 11, and 12 may be located in any region of the human spine, but typically are located in the thoracic or lumbar regions. In the illustrated embodiment, the upper vertebra 10 and the lower vertebra 12 are undamaged, while the intermediate vertebra 11 has experienced a burst fracture and, therefore, has been broken in multiple locations as shown at 11a. As a result of this burst fracture, the three vertebrae 10, 11, and 12 are arranged in an exaggeratedly curved or rounded orientation relative to one another, which is commonly referred to as kyphosis of the human spine. Although this invention will be described and illustrated in the context of re-aligning the vertebrae 10, 11, and 12 in a desired orientation to correct the kyphotic condition, it will be appreciated that this invention may be used to provide a desired orientation of any vertebrae in any desired region of the human spine.

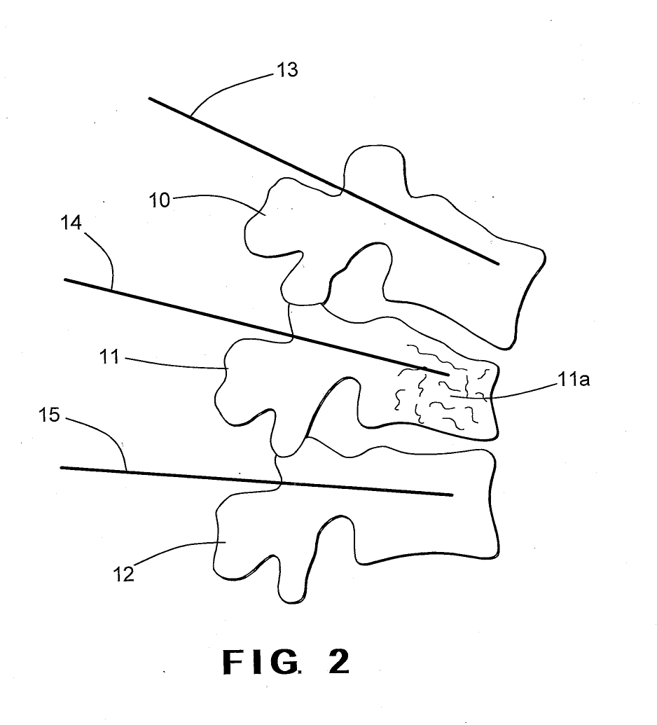

A first step of a method of this invention is illustrated in FIG. 2. As shown therein, guide wires 13, 14, and 15 are respectively inserted into the upper, intermediate, and lower vertebrae 10, 11, and 12. The guide wires 13, 14, and 15 are conventional in the art and may be embodied as any flexible members that are capable of being inserted within the upper, intermediate, and lower vertebrae 10, 11, and 12 for the purposes described herein. The guide wires 13, 14, and 15 may be formed from any desired material. The guide wires 13, 14, and 15 may be installed at any desired locations on the upper, intermediate, and lower vertebrae 10, 11, and 12 using any desired procedure. For example, the guide wires 13, 14, and 15 may be installed using a conventional Jamshidi needle (not shown) including a hollow outer cannula and an inner obturator having a tapered cutting edge. The procedure for such installation is described in co-pending application Ser. No. 12/535,026 filed Aug. 4, 2009, the disclosure of which is incorporated herein by reference.

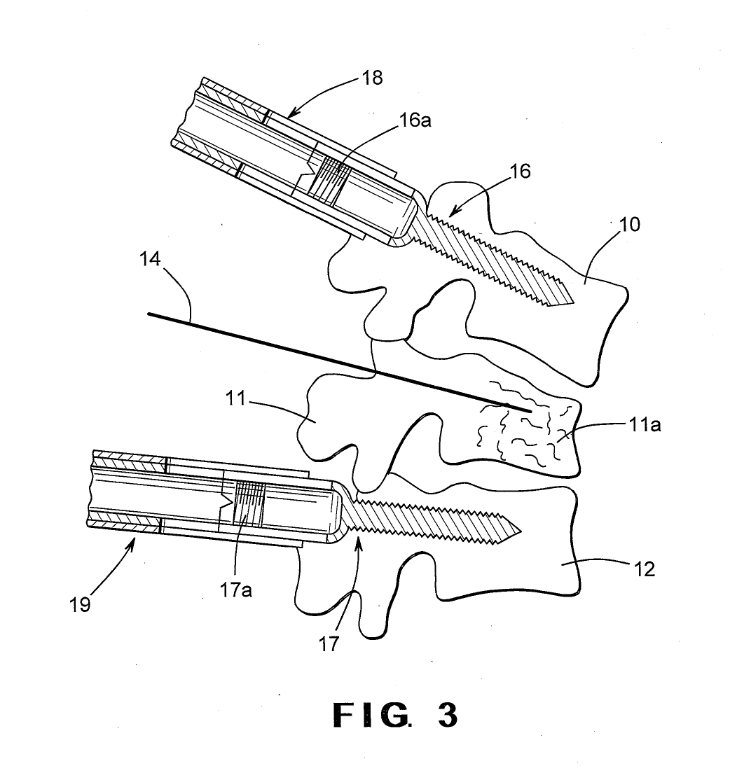

Next, as shown in FIG. 3, first and second pedicle screws 16 and 17 or similar vertebral fasteners are respectively inserted into the undamaged upper and lower vertebrae 10 and 12. Each of the first and second pedicle screws 16 and 17 is conventional in the art and includes a head portion and a threaded shank portion. The respective guide wires 13 and 15 may be used to facilitate the positioning of the first and second pedicle screws 16 and 17 at desired locations relative to the upper and lower vertebrae 10 and 12. To accomplish this, the first and second pedicle screws 16 and 17 may be cannulated so that they can be inserted over the trailing ends of the associated guide wires 13 and 15 and guided therealong to the desired locations on the upper and lower vertebrae 10 and 12.

The head portions of the first and second pedicle screws 16 and 17 can each include a generally U-shaped yoke portion and a driving mechanism. The illustrated yoke portions of the first and second pedicle screws 16 and 17 are internally threaded, as shown at 16a and 17a, or provided with other securement structures for a purpose that will be explained below. The shank portions of the first and second pedicle screws 16 and 17 can each be threaded into engagement with the associated one of the vertebrae 10 and 12. To accomplish this, respective insertion tube assemblies, indicated generally at 18 and 19, may be used. The insertion tube assemblies 18 and 19 are also conventional in the art and may, for example, be the same as disclosed in co-pending application Ser. No. 12/484,711, filed Jun. 15, 2009, the disclosure of which is also incorporated herein by reference.

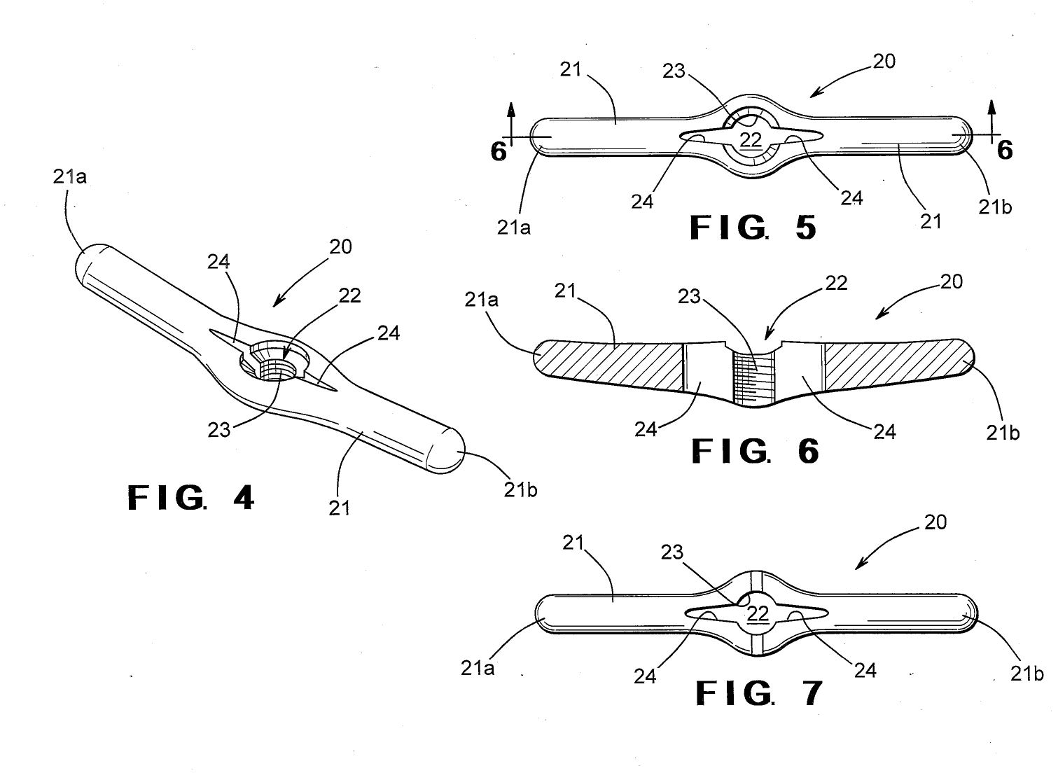

FIGS. 4 through 7 illustrated a first embodiment of a fixation rod, indicated generally at 20, in accordance with this invention. As shown therein, the fixation rod 20 includes a body 21 that is generally elongated and cylindrical in shape, having end portions 21a and 21b that are generally semi-spherical in shape. However, the body 21 of the fixation rod 20 may be formed having any desired shape. The illustrated fixation rod 20 is slightly curved from one end to the other, although such is not required. In the illustrated embodiment, the fixation rod 20 has a length that is suited for use in aligning the three vertebrae 10, 11, and 12 in the manner described below. However, the body 21 of the fixation rod 20 may have any desired length.

The body 21 of the fixation rod 20 may be formed from any desired material, such as titanium, stainless steel, and cobalt-chrome, and it may have any desired coating or finish. It may also be formed from other materials, such as PEEK, but preferably is formed from a material that is relatively rigid and is suited for internal use within the human body. If a radiolucent material such as PEEK is utilized, it may be useful in targeting of the pedicle of the fractured vertebra after provisional positioning of the longitudinal member through the anchors in the vertebral above and below. To further assist in pedicle targeting of the fractured vertebra, a metallic marker of varying thickness or geometry may be incorporated to define the outline of the central opening within the longitudinal member. This feature would assist in fluoroscopic localization of the central hole and its positioning over the fractured pedicle during surgery.

The body 21 of the fixation rod 20 has an aperture, indicated generally at 22, formed therethrough. In the illustrated embodiment, the aperture 22 is located at the center or midpoint of the body 21 of the fixation rod 20. However, the aperture 22 may be provided at any desired location on the body 21 of the fixation rod 20. In this first illustrated embodiment, the aperture 22 is generally circular in shape and defines a generally cylindrical inner wall having a helical thread or other retaining structure provided therein. However, the aperture 22 may be formed having any desired shape, and the helical thread 23 may be embodied as any structure that can function generally in the manner described below (and may, if desired, be omitted to provide a smooth inner wall for non-locking fixation in the fractured vertebra 11, as described below). In the illustrated embodiment, the aperture 22 further includes a pair of opposed, slit-like extensions 24. The illustrated extensions 24 are generally V-shaped and extend from opposite sides of the aperture 22 generally axially along the body 21 of the fixation rod 20. However, the extensions 24 may be provided having any desired size or shape, and a greater or lesser number of such extensions 24 (or none at all) may be provided as desired. The purposes for the aperture 22, the helical thread 23, and the extensions 24 will be explained below.

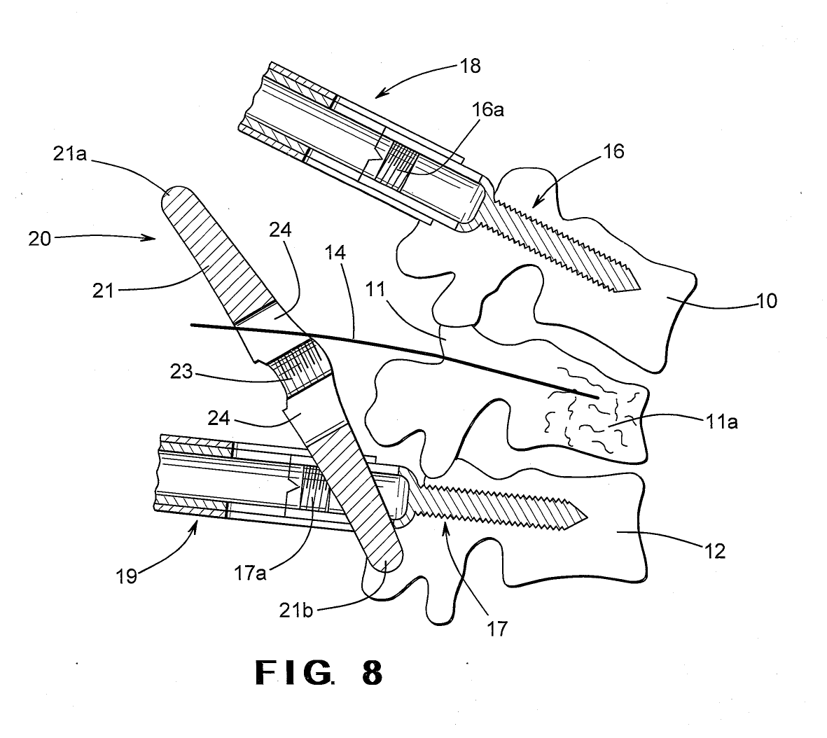

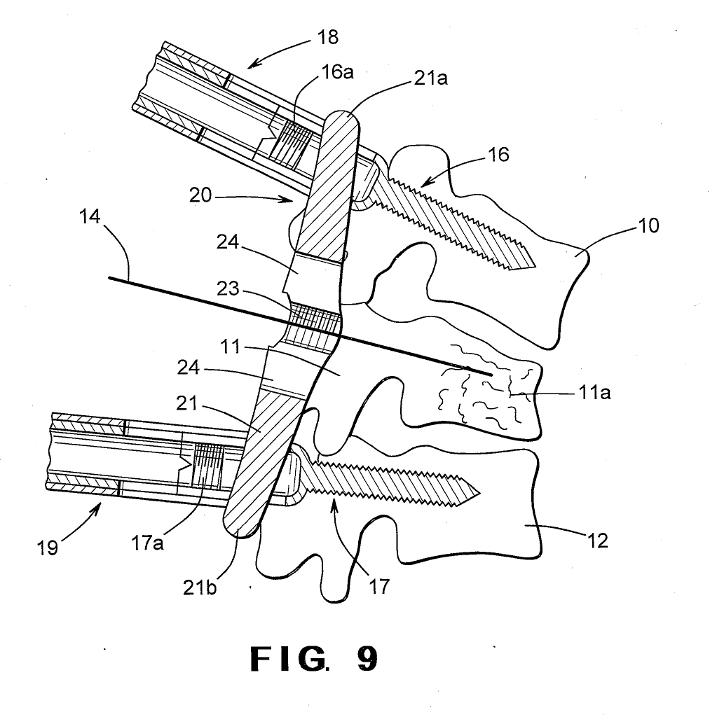

FIG. 8 shows the fixation rod 20 in the process of being installed on the first and second pedicle screws 16 and 17. As shown therein, the second end portion 21b of the fixation rod 20 is initially disposed within the yoke portion of the second pedicle screw 17 beneath the fascia and the muscle of the patient. At the same time, the aperture 22 is moved such that the second guide wire 14 is received therein. During such installation, the second guide wire 14 may be received within one or both of the slit-like extensions 24 of the aperture 22, thereby preventing undesired excessive angulation of the second guide wire 14 during the installation process. If desired, a conventional installation tool (not shown) may be connected to or otherwise used to facilitate the installation of the fixation rod 20 in this manner. Then, as shown in FIG. 9, the first end portion 21a of the fixation rod 20 is disposed within the yoke portion of the first pedicle screw 16. During this maneuver of delivering the proximal end of the fixation rod 16 into the yoke portion of the first pedicle screw 16, it may be necessary to split the overlying fascia and muscle of the patient. When the fixation rod 20 has been preliminarily installed, however, the first and second end portions 21a and 21b thereof are received within the yoke portions of the first and second pedicle screws 16 and 17, respectively, and the aperture 22 is aligned with the second guide wire 14 inserted with the intermediate vertebra 11.

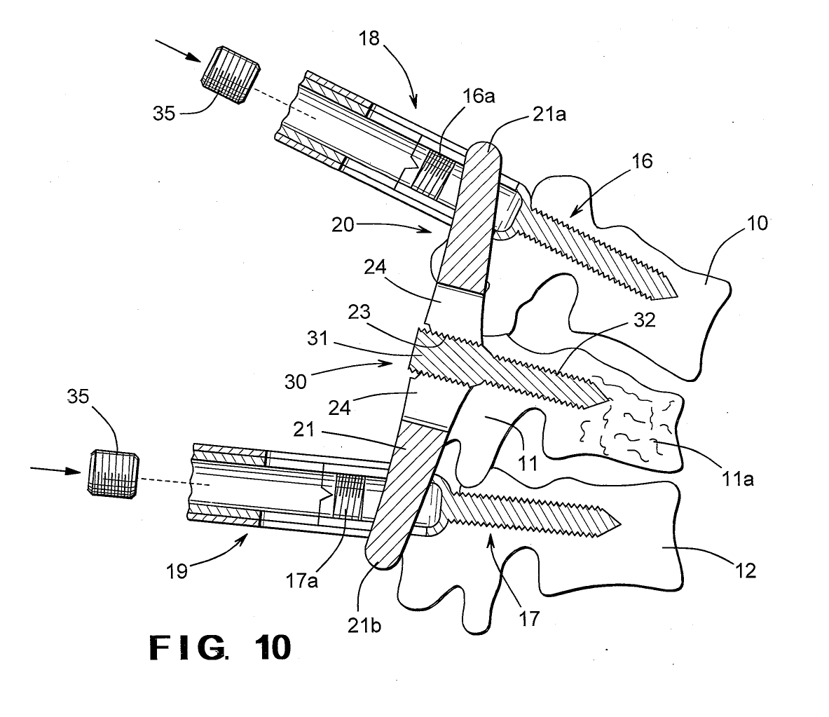

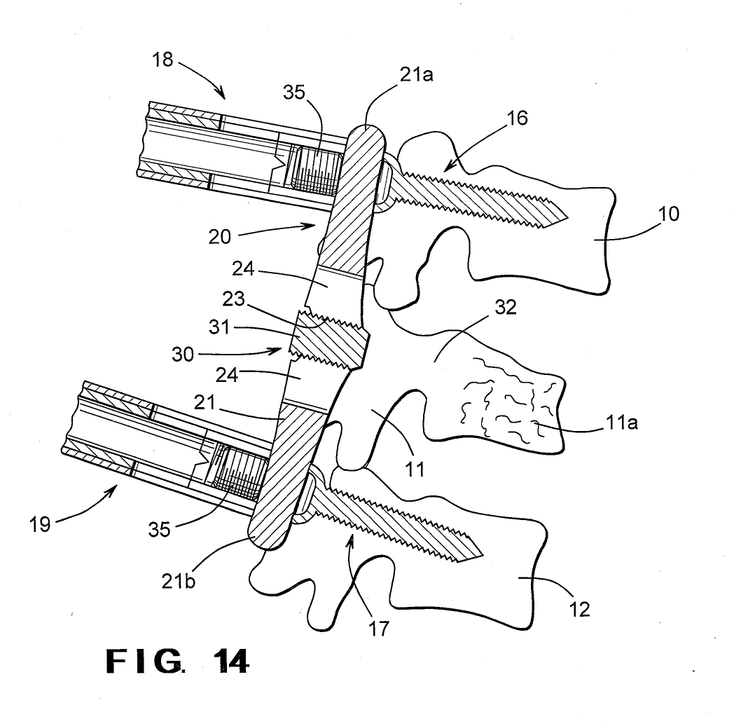

Next, as shown in FIG. 10, a vertebral fastener, such as a locking screw indicated generally at 30, is inserted into the intermediate vertebra 11 and secured to the fixation rod 20. The locking screw 30 can be used for this purpose when the intermediate vertebra 11 has not been damaged too severely. For example, the locking screw 30 can be used when the pedicle of the intermediate vertebra 11 has not been fractured apart from the remainder of such vertebra 11. If, however, the pedicle of the intermediate vertebra 11 has been fractured or if the intermediate vertebra 11 has been otherwise damaged too severely, then the use of the locking screw 30 can be omitted. In that instance, a filler screw (see FIG. 14 and description below) may be used in lieu thereof.

The locking screw 30 includes a head portion 31 and a shank portion 32. The head portion 31 of the locking screw 30 is generally cylindrical in shape and includes an outer surface having a helical thread or other retaining structure provided thereon that is adapted to cooperate with the helical thread 23 provided on the generally cylindrical inner wall defined by the aperture 22 formed through the fixation rod 20. The head portion 31 of the locking screw 30 also has a drive mechanism (not shown) provided therein that is adapted to cooperate with a conventional rotation tool (such as a conventional flat-head or hex-head screwdriver) to effect rotation of the locking screw 30 relative to the intermediate vertebra 11 and the fixation rod 20. The shank portion 32 of the locking screw 30 is also generally cylindrical in shape and includes an outer surface having a helical thread provided thereon. However, the locking screw 30 may be formed having any other desired configuration.

Rotation the locking screw 30 initially causes the shank portion 32 thereof to become engaged with the intermediate vertebra 11 in a known manner. Thereafter, further rotation of the locking screw 30 causes the head portion 31 thereof to become engaged with the fixation rod 20, as shown in FIG. 10. Thus, the intermediate vertebra 11, the locking screw 30, and the fixation rod 20 are all secured together. Preferably, the threads provided on the head and shank portions 31 and 32 of the locking screw 30 are formed in such a manner that the rotation of the locking screw 30 is not unduly inhibited when such threads are respectively engaged with fixation rod 20 and the intermediate vertebra 11.

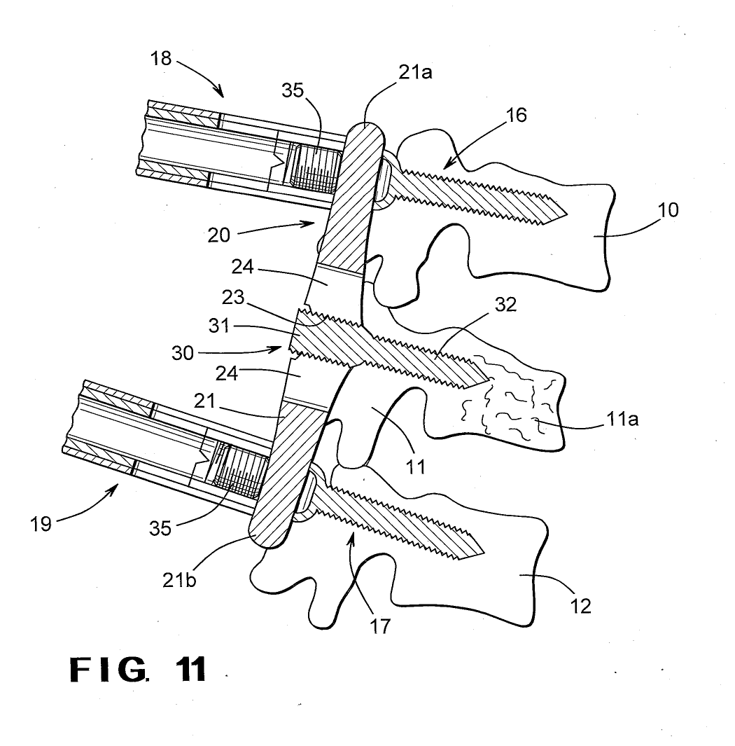

Next, the end portions 21a and 21b of the fixation rod 20 are respectively secured to the first and second pedicle screws 16 and 17. As shown in FIG. 11, this can be done by means of fasteners 35 that are secured to the securement structures provided on the yoke portions of the first and second pedicle screws 16 and 17. In the illustrated embodiment, the fasteners 35 are externally threaded and cooperate with the internally threaded portions 16a and 17a of the yoke portions of the first and second pedicle screws 16 and 17. The fasteners 35 are conventional in the art and are inserted through each of the insertion tube assemblies 18 and 19 (as shown in FIG. 10) into engagement with the yoke portions of the first and second pedicle screws 16 and 17. When secured thereto, the fasteners 35 move the first and second pedicle screws 16 and 17 into respective precise positions relative to the end portions 21a and 21b of the fixation rod 20. As a result, the upper and lower vertebrae 10 and 12 (to which the first and second pedicle screws 16 and 17 are attached) are positioned in accordance with the shape of the body portion 21 of the fixation rod 20, as also shown in FIG. 11. Such positioning corrects the kyphotic deformity of the three vertebrae 10, 11, and 12 and fixes them in a proper orientation relative to one another.

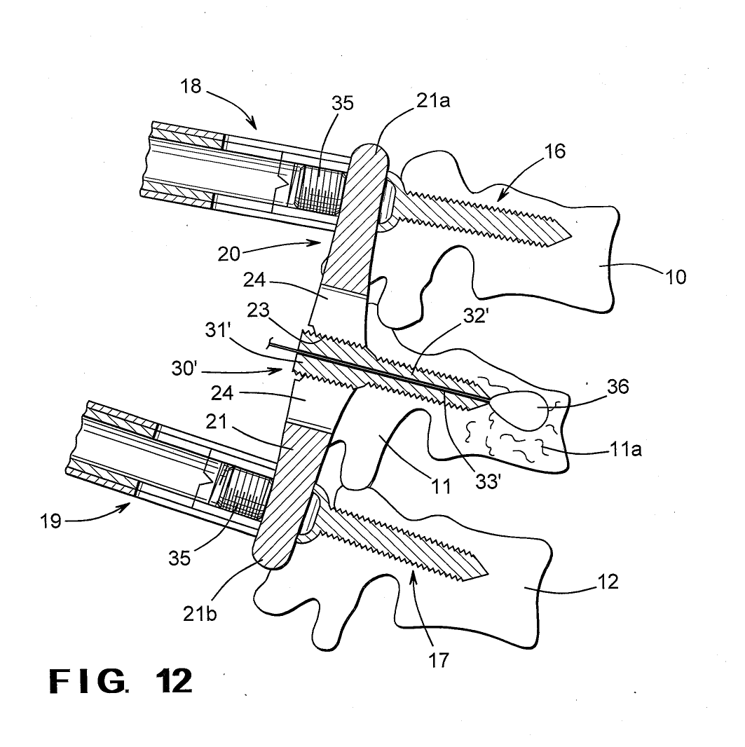

FIG. 12 is a schematic side elevational view similar to FIG. 11 showing a first alternative embodiment of a locking screw, indicated generally at 30′, in accordance with this invention. The locking screw 30′ is similar to the locking screw 30 described above and includes a head portion 31′ and a shank portion 32′. Additionally, however, the first alternative locking screw 30′ has an internal passageway 33′ formed therethrough that extends from the head portion 31′ to the shank portion 32′. The internal passageway 33′ can be used to facilitate access to the interior of the intermediate vertebra 11 and, in particular, to the region of the burst fracture 11a of such vertebra 11. For example, the internal passageway 33′ can be used to allow a balloon 36 to be inserted through the alternative locking screw 30′ and inflated within the region of the burst fracture 11a of the intermediate vertebra 11. The balloon 36 can be used to achieve additional correction of the alignment of the three vertebrae 10, 11, and 12 and to create a void within the intermediate vertebra 11. This may be followed by insertion of a particulate bone graft, bone graft substitute, mesh, or bone cement (preferably resorbable).

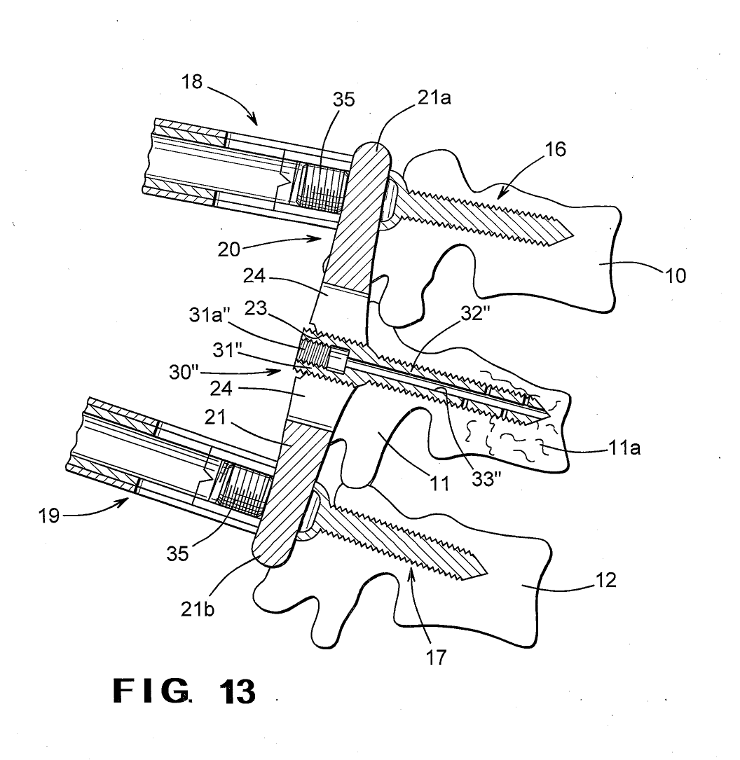

If desired, however, the bone graft or other material my be injected within the intermediate vertebra 11 without the prior use of the balloon 36. FIG. 13 is a schematic side elevational view similar to FIG. 11 showing a second alternative embodiment of a locking screw, indicated generally at 30″, in accordance with this invention that can be used for this purpose. The locking screw 30″ is similar to the locking screw 30′ described above and includes a head portion 31″ and a shank portion 32″. Additionally, however, the second alternative locking screw 30″ has an internal passageway 33″ formed therethrough that extends longitudinally from the head portion 31″ to the shank portion 32″. In the illustrated embodiment, the internal passageway 33″ includes a plurality of transversely extending passageways that provide communication from the longitudinal portion of the internal passageway 33″ to the region of the burst fracture 11a of the vertebra 11. The internal passageway 33″ can be used to facilitate access to the interior of the intermediate vertebra 11 and, in particular, to the region of the burst fracture 11a of such vertebra 11 for the insertion of a particulate bone graft, bone graft substitute, mesh, or bone cement (preferably resorbable), as described above. To facilitate the insertion of this material, the head portion 31″ of the locking screw 30″ may have a counterbore region, such as shown at 31a″, that can be used to facilitate the connection of a fitting (not shown) of a conventional tool for the insertion of this material. As shown in FIG. 13, the counterbore region 31a″ may be internally threaded or otherwise structured to facilitate the connection of the fitting thereto.

Pedicle screws of prior art usually require injection of bone cement or similar material prior to installation of the longitudinal member and before full height restoration of the fractured vertebra by ligamentotaxis. This often leads to undercorrection of the deformity. The disclosed invention facilitates insertion of the longitudinal member and restoration of the height of the fractured pedicle before injection of bone cement or similar material, leading to improved correction and ability to inject more material, particularly osteogenic material.

FIG. 14 is a schematic side elevational view similar to FIG. 11 of a filler screw or similar mechanism, indicated generally at 40, that can be used in lieu of the locking screws 30 and 30′ in accordance with this invention. As mentioned above, the locking screws 30 and 30′ can be used when the intermediate vertebra 11 has not been damaged too severely. If, however, the pedicle of the intermediate vertebra 11 has been fractured or if the intermediate vertebra 11 has been otherwise damaged too severely, then the locking screws 30 and 30′ cannot be used. In those instances, the filler screw 40 can be threaded into engagement with the fixation rod 20. The filler screw 40 is generally cylindrical in shape and includes an outer surface having a helical thread provided thereon that is adapted to cooperate with the helical thread 23 provided on the generally cylindrical inner wall defined by the aperture 22 formed through the fixation rod 20. The filler screw 40 also has a drive mechanism (not shown) provided therein that is adapted to cooperate with a conventional rotation tool (such as a conventional flat-head or hex-head screwdriver) to effect rotation of the filler screw 40 relative to the fixation rod 20. Although the filler screw 40 does not secure the intermediate vertebra 11 to the fixation rod 20, it is effective to increase the overall strength of the fixation rod 20 for subsequent attachment to the upper and lower vertebrae 10 and 12, as described above. Alternatively, the aperture 22 formed through the fixation rod 20 can be omitted entirely, which would eliminate the need for the filler screw 40.

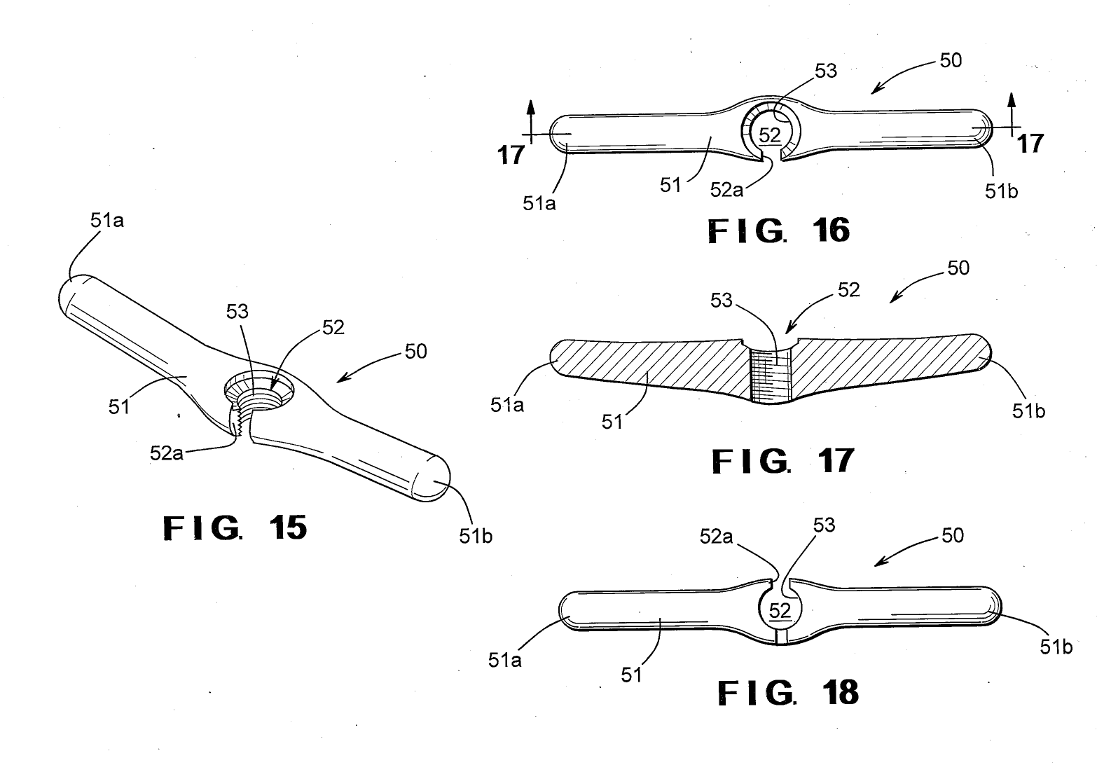

FIGS. 15 through 18 illustrated a second embodiment of a fixation rod, indicated generally at 50, in accordance with this invention. As shown therein, the fixation rod 50 includes a body 51 that is generally elongated and cylindrical in shape, having end portions 51a and 51b that are generally semi-spherical in shape. However, the body 51 of the fixation rod 50 may be formed having any desired shape. The illustrated fixation rod 50 is slightly curved from one end to the other, although such is not required. In this second illustrated embodiment, the fixation rod 50 has a length that is suited for use in aligning the three vertebrae 10, 11, and 12 in the manner described below. However, the body 51 of the fixation rod 50 may have any desired length. The body 51 of the fixation rod 50 may be formed from any desired material, but preferably is formed from a material that is relatively rigid and is suited for internal use within the human body.

The body 51 of the fixation rod 50 has an aperture, indicated generally at 52, formed therethrough. In the illustrated embodiment, the aperture 52 is located at the center or midpoint of the body 51 of the fixation rod 50. However, the aperture 52 may be located at any desired location on the body 51 of the fixation rod 50. In this second illustrated embodiment, the aperture 52 is generally circular in shape, but is not completely circular. Rather, the body 51 of the fixation rod 50 has a slot 52a formed therethrough that communicates with the interior of the aperture 52. The slots 52a may be kept medially or laterally depending on patient anatomy and location of the second guide wire 14. The aperture 52 nonetheless defines a generally cylindrical inner wall having a helical thread 53 provided therein. However, the aperture 52 may be formed having any desired shape, and the helical thread 53 may be embodied as any structure that can function generally in the manner described below.

As discussed above, when the first embodiment of the fixation rod 20 is installed on the first and second pedicle screws 16 and 17, the aperture 22 formed therethrough is moved such that the second guide wire 14 is received therein. In contrast, when the second embodiment of the fixation rod 50 is installed on the first and second pedicle screws 16 and 17, the slot 52a allows the second guide wire 14 to pass laterally therethrough into the aperture 52, thus facilitating the installation of the fixation rod 50. Otherwise, the second embodiment of the fixation rod 50 functions in the same manner as the first embodiment of the fixation rod 20.

If desired, a locking feature (not shown) may be provided on the second embodiment of the fixation rod 50 to selectively open and close the slot 52a. Also, if desired, a collar or a washer (not shown) may be utilized under the head portion 31 of the locking screw 30, 30′, or 30″ to bridge the lateral or medial opening on the fixation rod 20 as the locking screw 30 is seated in to the aperture 52. Additionally, the locking screw 30, 30′, or 30″ or the filler screw 40 may be provided with a locking feature (not shown) to positively retain it on the fixation rod 20 or 50.

The above detailed description of this invention is given for explanatory purposes. It will be apparent to those skilled in the art that numerous changes and modifications other than those cited can be made without departing from the scope of the invention. Accordingly, the whole of the foregoing description is to be construed in an illustrative and not a limitative sense, the scope of the invention being defined by the appended claims.

What is claimed is:

1. A spinal fixation device comprising:
a fixation rod including a body having an aperture formed therethrough;
first and second vertebral fasteners secured to the body and adapted to be secured to respective vertebrae; and
a third vertebral fastener extending through the aperture in the body of the fixation rod and adapted to be secured to a vertebra.

2. The spinal fixation device defined in claim 1 wherein the aperture defines an inner surface having a retaining structure provided thereon, and wherein the third vertebral fastener cooperates with the retaining structure.

3. The spinal fixation device defined in claim 2 wherein the retaining structure is a helical thread.

4. The spinal fixation device defined in claim 1 wherein the aperture defines an inner surface having a smooth surface provided thereon, and wherein the third vertebral fastener does not lockingly cooperate with the aperture.

5. The spinal fixation device defined in claim 1 wherein the aperture is complete.

6. The spinal fixation device defined in claim 5 further including an extension that extends from the aperture.

7. The spinal fixation device defined in claim 5 further including a pair of extensions that extend from opposite sides of the aperture.

8. The spinal fixation device defined in claim 1 wherein the aperture is incomplete.

9. The spinal fixation device defined in claim 8 wherein the body of the fixation rod has a slot formed therethrough that communicates with the aperture to make the aperture incomplete.

10. The spinal fixation device defined in claim 9 wherein the opening is medial or lateral.

11. The spinal fixation device defined in claim 9 further including a washer or collar disposed within the aperture to bridge the slot.

12. The spinal fixation device defined in claim 1 wherein the fixation body is thicker in a central portion than in end portions.

13. The spinal fixation device defined in claim 1 further including a filler mechanism that is provided within the aperture.

14. The spinal fixation device defined in claim 13 wherein the aperture defines an inner surface having a retaining structure provided thereon, and wherein the filler mechanism cooperates with the retaining structure.

15. The spinal fixation device defined in claim 14 wherein the retaining structure is a helical thread.

16. The spinal fixation device defined in claim 1 wherein the third vertebral fastener has a passageway formed therethrough from a head portion to a shank portion.

17. The spinal fixation device defined in claim 16 further including a balloon extending through the passageway.
